(19) United States
(12) Patent Application Publication
(10) Pub. No.: US 2017/0175080 A1
(43) Pub. Date: Jun. 22, 2017

(54) COMPOSITIONS AND METHODS FOR CELL CULTURE

(71) Applicant: SAN DIEGO BLOOD BANK, San Diego, CA (US)

(72) Inventors: James BARLOW, San Diego, CA (US); Will DAVEY, San Diego, CA (US); Robert TRESSLER, San Diego, CA (US)

(21) Appl. No.: 15/382,803

(22) Filed: Dec. 19, 2016

Related U.S. Application Data
(60) Provisional application No. 62/268,619, filed on Dec. 17, 2015.

(51) Int. Cl. C12N 5/078; A61K 9/00; A61K 35/19; A61K 8/98; A61K 8/36; A61K 38/44; A61K 8/66; A61K 31/375; A61K 8/67; A61K 31/201; C12N 9/02; A61Q 19/08

(52) U.S. Cl. CPC C12N 5/0644; C12N 9/0089; C12Y 115/01001; A61K 9/0014; A61K 35/19; A61K 8/983; A61Q 19/08; A61K 38/446; A61K 8/66; A61K 31/375; A61K 8/676; A61K 31/201; A61K 8/361; A61K 2800/48

(57) ABSTRACT

A human platelet lysate preparation that contains human plasma is provided. This preparation can be generated from human platelets by concentrating and washing them under defined conditions to control the degree of human plasma present. The washed platelet concentrate is then subjected to a freeze-thaw cycle to produce platelet lysate which is centrifuged and filtered through 0.65μ, 0.45μ and 0.2μ filters, aliquoted and stored frozen at <−20° C. until thawed for use. This invention describes the novel finding that the controlled addition of human plasma to the lysate preparation significantly enhances the cell growth potency of the lysate preparation. This lysate can be used as a media supplement to replace fetal bovine serum (FBS) or other non-human serum additives used for the culture of mammalian cells. This invention also describes the formulation and use of the lysate preparation as a topical application for skin care, and wound healing, including anti-wrinkling, anti-scarring and wound resolution applications of the invention.

### CROSS-REFERENCE TO RELATED APPLICATIONS

The present claims priority to, and the benefit of U.S. Provisional Patent Application Ser. No. 62/268,619, filed Dec. 17, 2015, which is hereby incorproated by reference in its entirety for all purposes.

### FIELD OF THE INVENTION

The present invention is related to compositions and methods of using the same. In some embodiments, the invention provides a platelet lysate preparation and process with enhanced cell growth potency for use as a media supplement for the growth of mammalian cells. In other embodiments the invention provides a human platelet lysate formulation for topical application to skin for wound healing, scar and wrinkle reduction or prevention, and methods of using the same.

### BACKGROUND OF THE INVENTION

The current focus in biotechnology on the production of cell-based therapies for the treatment of various diseases has triggered a critical need to find an alternative to fetal bovine serum (FBS) for the growth and expansion of cell therapies. Human platelet lysate has been shown to be an attractive alternative.

One issue for human platelet lysate preparations used for cell culture is the variability in the growth promoting potency of various platelet lysate preparations. A number of factors can play a role in this, including variations in platelet pool size, protein concentration, additives, such as heparin or other anticoagulants, processing methods and the amount of plasma or serum components that may be present in the final lysate product.

To date little or no studies to systematically evaluate the role of these potential variables has been carried out. Therefore, there remains a need in the art for producing platelet lysate preparations having high gorwth promoting potency.

### SUMMARY OF THE INVENTION

This invention relates to the compositions comprising mammalian platelet lysate, methods for preparing the compositions, and methods for using the compositions to culture cells.

The present invention provides mammalian platelet lysate compositions. In some embodiments, the compositions comprise lysed mammalian platelets and mammalian plasma. In some embodiments, the concentration of mammalian plasma in the composition is less than about 30%, less than about 20%, or less than about 10% of the total volume. In some embodiments, the concentration of mammalian plasma in the composition is from about 1% to about 10%.

In some embodiments, the compositions have concentrated platelets. In some embodiments, the amount of platelets in the compositions is platelet equivalents of from about 20×10¹¹ to 200×10¹¹ per liter.

In some embodiments, the mammalian platelet lysate composition is derived from human.

In some embodiments, the composition is substantially free of mammalian platelet membranes.

In some embodiments, the lysed mammalian platelets are derived from a single donor platelet unit, or derived from multiple donor platelet units.

In some embodiments, lysed mammalian platelets are derived from about 5 to 40 donor platelet units.

In some embodiments, the composition is substantially free of any heparin.

In some embodiments, the concentration of platelet lysates in the composition is at least about 70%, at least about 80%, or at least about 90%. In some embodiments, the concentration of platelet lysates is from about 90% to about 99%.

In some embodiments, the composition is sterile.

In some embodiments, the composition is derived from multiple donor platelet units with or without matching blood type.

In some embodiments, the composition is derived from multiple donor platelet units produced from donors under the age of 45 years.

In some embodiments, the composition has increased growth-promoting activity on cells when used as a supplement in a cell culture media, compared to a plasma-free platelet lysate composition. In some embodiments, the composition has at least 50% of increased growth-promoting activity on cells compared to a plasma-free platelet lysate composition.

In some embodiments, the composition has increased growth-promoting activity on cells when used as a supplement in a cell culture media, compared to fetal bovine serum (FBS) at the same concentration. In some embodiments, the composition has at least 20% of increased growth promoting activity on cells compared to fetal bovine serum (FBS) at the same concentration.

In some embodiments, the composition is capable of maintaining an increased cell density when used in a cell culture media, compared to a plasma-free platelet lysate composition, or fetal bovine serum (FBS) at the same concentration. In some embodiments, the composition is capable of maintaining at least 50%, at least 60%, at least 70%, at least 80%, at least 90%, at least 95%, or more increased cell density at a given time point, such as the 120-144 hr time points.

In some embodiments, the composition shortens cell growth doubling time when used as a supplement in a cell culture media compared to a cell culture media without the composition. In some embodiments, the cell growth doubling time was shortened to no more than 25-35 hours, depending on cell types. In some embodiments, the cell growth doubling time was shortened by at least 5%, at least 10%, at least 15%, at least 20%, at least 25%, at least 30%, at least 35%, at least 40%, at least 45%, at least 50%, or more, depending on cell types.

The present invention also provides formulations. In some embodiments, the formulations comprise the platelet lysate compositions of the present invention. In some embodiments, the platelet lysate is a mammalian platelet lysate, such as human platelet lysate.

In some embodiments, the concentration of platelet lysates in the formulations is from about 1% to about 10%.

In some embodiments, the formulations comprise mammalian platelet lysate and mammalian plasma. In some embodiments, the platelet lysate concentration in the formulations is from about 1% to about 10%. In some embodiments, the mammalian plasma concentration in the formulations is from about 0.01% to 1%.

In some embodiments, the formulations are growth media. In some embodiments, the formulations are for topical application to skin.

In some embodiments, the formulations for topical application further comprise at least one antioxidant. In some embodiments, the antioxidant is Superoxide Dismutase (EC 1.15.1.1). In some embodiments, the Superoxide Dismutase is SOD1 (Superoxide Dismutase (Cu—Zn)), SOD2 (MnSOD), or SOD3 (extracellular superoxide dismutase).

In some embodiments, the formulations for topical application further comprise at least one additional active skin care agent. In some embodiments, the additional active skin care agent is vitamin moiety. In some embodiments, the vitamin moiety is a vitamin C moiety. In some embodiments, the vitamin C moiety is ascorbate or derivatives thereof. In some embodiments, the vitamin C moiety is an anhydrous formulation of ascorbic acid, including but not limited to tetrahexyldecyl ascorbate and ascorbyl tetraisopalmitate.

In some embodiments, the formulation for topical application is in a cream. In some embodiments, the formulation is in a serum.

In some embodiments, the formulations for topical application further comprise at least one emollient and/or thickening agent. In some embodiments, the emollient and/or thickening agent is natural, synthetic, or semi-synthetic. In some embodiments, the emollient and/or thickening agent comprises a compound selected from the group consisting of polysaccharides, proteins, alcohols, silicones, fatty acids, and waxes. In some embodiments, the emollient and/or thickening agent comprises linoleic acid or derivatives thereof.

The present invention also provides methods of promoting cell growth. In some embodiments, the methods comprise growing cells in a formulation of the present invention described herein.

In some embodiments, the cells are human cells.

In some embodiments, the cells are stem cells. In some embodiments, the stem cells are pluripotent hematopoietic stem cells, pluripotent mesenchymal stem cells, committed progenitor hematopoietic stem cells, including myeloid, lymphoid and erythroid lineages, committed progenitor mesenchymal stem cells, including cardio, neuro, hepatic, pancreatic, pulmonary and musculo-skeletal cells, connective tissue cells, cord blood and cord tissue-derived stem cells, embryonic stem cells, committed progenitors derived from embryonic stem cells, and induced pluripotent stem cells.

In some embodiments, the cells are tumor cells. In some embodiments, the cells are malignant solid tumor cells and solid tumor stem cells. In some embodiments, the cells are malignant hematological cells and malignant hematological stem cells.

In some embodiments, the cells are normal cells. In some embodiments, the cells are disease-associated cell types.

In some embodiments, the genotypic and phenotypic properties of cells are maintained when the cells are cultured in the media of the present invention.

In some embodiments, the cells are skin cells. In some embodiments, the skin cells are dermal fibroblasts.

In some embodiments, the growth media used for cell culture are substantially free of fetal bovine serum (FBS) or other non-human animal product additives. In some embodiments, the methods comprise topical application of a formulation of the present invention to the skin of a subject in need, such as subjects with skin having, had, or at risk of having wounds, scars, and/or wrinkles.

In some embodiments, the formulations for topical application can be used to treat wounds, scars, and/or wrinkles. In some embodiments, the treatment has a statistically significant effect.

In some embodiments, mammalian platelet lysate compositions of the present invention are produced by a freeze-thaw process and subsequent cold processing by centrifugation and filtration.

In some embodiments, the process comprises controlling presence of human plasma.

In some embodiments, to reduce the variability of the potency of heparin-binding growth factors present in the platelet lysate, the process does not include heparin as an additive to the product.

### BRIEF DESCRIPTION OF THE DRAWINGS

FIG. 1 depicts growth-promoting activity on MSCs of media prepared with a final concentration of 5% human platelet lysate containing human plasma, compared to 5% FBS.

FIG. 2 depicts growth-promoting activity on MSCs of media prepared with a final concentration of 2% human platelet lysate containing human plasma, and its ability of maintaining higher cell densities, when compared to 2% FBS or 2% Clearsate containing no plasma.

FIG. 3 depicts doubling times for A549 lung adenocarcinoma cells in media prepared with a final concentration of 2% human platelet lysate containing human plasma (2% CS+1W), compared to original Clearsate, Mill Creek human platelet lysate, Cook Platelet lysate and FBS.

FIG. 4 depicts growth-promoting activity on primary human dermal fibroblasts of media prepared with a final concentration of 2% human platelet lysate containing human plasma, and its ability of maintaining higher cell densities, when compared to 2% FBS or 2% Clearsate containing no plasma.

### DETAILED DESCRIPTION OF THE INVENTION

Compositions of the present invention comprise platelets lysate. Platelets, or thrombocytes, are cells that do not have a nucleus containing DNA derived from fragmentation of precursor megakaryocytes. Platelets circulate in the blood of mammals and are involved in hemostasis, leading to the formation of blood clots.

In some embodiments, the concentration of platelet lysates in the composition is from about 80%, about 85%, about 90%, about 91%, about 92%, about 93%, about 94%, about 95%, about 96%, about 97%, about 98%, about 99% or more.

In some embodiments, the amount of equivalent platelets in the composition is about 20×10¹¹ to 200×10¹¹ per liter, such as about 20×10¹¹ per liter, about 30×10¹¹, about 40×10¹¹ per liter, about 50×10¹¹ per liter, about 60×10¹¹ per liter, about 70×10¹¹ per liter, about 80×10¹¹ per liter, about 90×10¹¹ per liter, about 100×10¹¹ per liter, about 110×10¹¹ per liter, about 120×10¹¹ per liter, about 130×10¹¹ per liter, about 140×10¹¹ per liter, about 150×10¹¹ per liter, about 160×10¹¹ per liter, about 170×10¹¹ per liter, about 180×10¹¹ per liter, about 190×10¹¹ per liter, or about 2000×10¹¹ per liter.

In some embodiments, the platelet lysates of the present invention is lyophilized. As used herein, the terms “lyophilization,” lyophilize,” or the like refer to a freeze-drying or dehydration process that is often used to preserve platelets. In some embodiments, lyophilization is used primarily not just as a preservative process, but rather, to further lyse platelets after initial freeze-thaw or other lysis technique is conducted. In other words, in accordance with examples of the present disclosure, after lysates are formed as described herein, lyophilization provides the added benefit of preserving the growth factors, cytokines, chemokines, and other contents initially enclosed within or bound to the surface the platelets, but which are released when platelets are lysed as described herein. The process typically works by freezing the material and reducing surrounding pressure to allow frozen water in the material to sublimate directly from the solid phase to the gas phase.

The compositions of the present invention further comprise plasma. Blood plasma is the pale yellow liquid component of blood that normally holds the blood cells in whole blood in suspension. In some embodiments, it is mostly water (up to 95% by volume), and contains dissolved proteins (6-8%) (such as serum albumins, globulins, and fibrinogen), glucose, clotting factors, electrolytes (Na+, Ca²⁺, Mg²⁺, HCO₃⁻, Cl⁻, etc.), hormones, and/or carbon dioxide. In some embodiments, the plasma is isolated from blood of a mammal. In some embodiments, the plasma is a simulated body fluid (SBF), such as solutions having a similar ion concentration to that of mammalian blood plasma.

In some embodiments, the concentration of mammalian plasma in the composition is about 20% of the total volume or less. In some embodiments, the concentration is about 20%, about 15%, about 20%, about 15%, about 10%, about 9%, about 8%, about 7%, about 6%, about 5%, about 4%, about 3%, about 2%, about 1%. In some embodiments, the mammalian plasma in the composition is about 1% to about 10%. As used herein, the term “about” is used to provide flexibility to a numerical range endpoint by providing that a given value may be “a little above” or “a little below” the endpoint. The degree of flexibility of this term can be dictated by the particular variable and would be within the knowledge of those skilled in the art to determine based on experience and the associated description herein.

In some embodiments, the compositions are substantially free of mammalian platelet membranes and/or other cell debris. As used herein, substantially free means that there is only non-detectable amount of platelet membranes, or the amount of platelet membranes cannot be further reduced by any reasonable, commercially sound method, or the amount of platelet membranes do not substantially interfere with the use of the compositions. Platelet membranes can be separated from the compositions using any suitable methods known in the field, such as density gradient.

The compositions of the present invention may contain one or more anticoagulant to prevent coagulation. As used herein, the term “anticoagulant” refers to compositions that inhibit clotting when concentrating or collecting platelets for use in accordance with examples of the present disclosure. Anticoagulants generally are available as inhibitors of clotting factor synthesis, inhibitors of thrombin, or antiplatelet drugs. Inhibitors of clotting factor synthesis that inhibit the production of certain clotting factors in the liver, include compositions such as warfarin (Coumadin). Inhibitors of thrombin interfere with blood clotting by blocking the activity of thrombin, and include compositions such as heparin and lepirudin (Refludan). Antiplatelet drugs interact with platelets themselves, and include drugs such as aspirin, ticlopidine (Ticlid), clopidogrel (Plavix), tirofiban (Aggrastat), eptifibatide (Integrilin), etc. In some embodiments, the anticoagulant does not contain heparin. In some embodiments, the compositions of the present invention are substantially free of fibrinogen, cryoprecipitate, and/or thrombin. Examples of anticoagulants include a coumarin (e.g., warfarin), a synthetic pentasaccharide inhibitor of factor Xa (e.g., fondaparinux, idraparinux), a direct factor Xa inhibitor, (e.g., rivaroxaban, apixaban, and edoxaban), and a direct thrombin inhibitor (e.g., hirudin, lepirudin, bivalirudin, argatroban and dabigatran). In some embodiments, the anticoagulant is a calcium chelating anticoagulant, such as a citrate.

In some embodiments, the composition is sterile or substantially free of pathogens. For example, the composition is substantially free of bacteria, fungi, and/or virus. In some embodiments, the pathogens are removed from the composition by filtration, or other suitable methods known in the field.

In some embodiments, the compositions of the present invention are substantially free of fetal bovine serum (FBS) or other non-human animal product additives.

The compositions of the present invention have high growth-promoting activity when used in a cell culture media, compared to a platelet lysate composition substantially free of plasma, or compared to fetal bovine serum (FBS) at the same concentration. In some embodiments, the compositions of the present invention can increase at least 10%, at least 20%, at least 30%, at least 40%, at least 50%, at least 60%, at least 70%, at least 80%, at least 90%, or more growth-promoting activity on cells compared to a plasma-free platelet lysate composition at the same concentration. In some embodiments, the compositions of the present invention can increase at least 5%, at least 10%, at least 15%, at least 20%, at least 25%, at least 30%, or more growth-promoting activity on cells compared FBS at the same concentration.

The compositions of the present invention are capable of maintaining a high cell density at a given time point when used as a supplement to a cell culture media. In some embodiments, the compositions of the present invention can maintain at least 10%, at least 20%, at least 30%, at least 40%, at least 50%, at least 60%, at least 70%, at least 80%, at least 90%, or more of increased cell density at the 0-24, 24-48, 48-72, 72-96, 96-120, or 120-144 hr time points when used in a cell culture media compared to a plasma-free platelet lysate composition, or fetal bovine serum (FBS) at the same concentration.

The compositions of present invention shortens cell growth doubling time when used as a supplement in a cell culture media compared to a cell culture media without the composition. In some embodiments, depending on the cell types, the cell growth doubling time is shortened to no more than about 24 hours, 25 hours, 26 hours, 27 hours, 28 hours, 29 hours, 30 hours, 31 hours, 32 hours, 33 hours, 34 hours, 35 hours, 36 hours, 37 hours, 38 hours, 39 hours, 40 hours, 41 hours, 42 hours, 43 hours, 44 hours, 45 hours, 46 hours, 47 hours, 48 hours. In some embodiments, the cell growth doubling time is shortened by at least 5%, at least 10%, at least 15%, at least 20%, at least 25%, at least 30%, at least 35%, at least 40%, at least 45%, at least 50%, or more when grown in a cell culture media comprising the compositions of present invention compared to a cell culture media comprising a platelet lysate composition substantially free of plasma, or FBS.

To produce the compositions of the present invention, many methods can be used. In some embodiments, the compositions of the present invention can be sourced from normal healthy donors. In some embodiments, the donors have passed necessary infectious disease testing.

In some embodiments, the compositions are derived from mammalian blood. Depending on the purpose, a suitable mammal can be chosen as the blood source. In some embodiments, the mammal is human. The mammalian animals which are intended used according to the present invention can in principle be all animals, young or adult, from which the required amount of platelets can reasonably be collected. In some embodiments, mainly adult mammalian animals are used. Examples of non-human animals are slaughter animals and other farmbred animals such as cattle, pigs, sheep or poultry. In some embodiments, blood donors are healthy and satisfy the requirements set forth by relevant regulatory agencies. For example, the blood can satisfy the requirements which a national food/drug administration places on products intended for foodstuffs or medical use, or satisfy the corresponding regulations within the EEC and the US. The present cell culture medium can, wholly or partly, replace fetal bovine serum.

In some embodiments, the donors are human. In some embodiments, the donors are under the age of about 50, about 45, about 40, or less.

In some embodiments, the compositions are derived from multiple donor platelet units. The multiple donor platelet units can either have or do not have matching blood type.

Platelet lysate and plasma can be produced by methods described in the present invention, or any suitable methods known to one skilled in the art. In accordance with the present disclosure, a “lysate” is the composition prepared where platelets are destroyed by disrupting their cell membrane. This can be done chemically, mechanically, by liquid homogenization, sonication, or other methods known in the field.

In some embodiments, freeze-thaw lysates can be formed by freezing a platelet suspension and then thawing the material, though other freeze-thaw regimens are also included, provided they lead to cytolysis of the platelets. With the freeze-thaw technique, this method causes cells to swell and break as ice crystals form, followed by contraction at thawing. Thus, the cyclical swelling and contracting ultimately causes the platelets to break open. Multiple cycles are typically used for more complete lysis, but the “more complete” lysis is not necessarily required. Varying degrees of platelet cytolysis can occur, e.g., at least 30%, at least 50%, at least 70%, at least 90%, or up to 100% cytolosis, by platelet count.

In some embodiments, platelet lysate can be generated from single or pooled donor-donated platelets isolated from whole blood or by apheresis. In some embodiments, the apheresis is based on separating substances having different densities, such as centrifugation. In some embodiments, the apheresis involves absorption onto beads coated with an absorbent material and filtration. In some embodiments, centrifugation process is controlled to selectively to remove desired components. The variables of centrifugation process for determining the centrifugation include spin speed, bowl diameter, sit time, solutes added, plasma volume, and cellular content of the donor.

Different processes of making platelet lysate can be used. In some embodiments, the process includes freezing isolated platelets at low temperatures and thawing. In some embodiments, the process is repeated two or more times to cause complete platelet lysis. In some embodiments, other methods involving physical, chemical, biological process for isolating platelet lysate, or a combination thereof can be used.

The lysed mammalian platelets can be derived from a single donor platelet unit or multiple donor platelet units. For example, when the lysed mammalian platelets can be derived from about 5, about 10, about 15, about 20, about 25, about 30, about 35, about 40, about 45, about 50, about 60, about 70, about 80, about 90, about 100, or more donor platelet units.

In some embodiments, platelets can be pooled from whole-blood or by a device that separates the platelets and returns other portions of the blood to a donor (e.g., apheresis). In some embodiments, in order to isolate platelets, serum is placed into a centrifuge for a “soft spin” in which the platelets remain suspended in the plasma. The platelet-rich plasma (PRP) is removed and then centrifuged at a faster setting to harvest the platelets from the plasma. In some embodiments, the collected platelets are chilled to slow the degradation of the cells. Platelet lysate can be obtained by platelet lysis. The platelet lysate may also include any medium in which the lysed platelets are contained. In some embodiments, freezing-thawing is used for platelet lysis. However, this is only a typical, but not the only way for lysing cells in this disclosure. In some embodiments, mechanical lysis, typically through the use of shear forces, can be used for producing a lysate. In some embodiments, lysis buffers, typically acting by placing the cells in a hypotonic solution, can also be used. In some embodiments, the lysis process may consist of any combination of such methods, or methods known in the field.

In some embodiments, the present disclosure provides compositions prepared by the process comprising: (1) Preparing a platelet concentrate (pre-lysate). In some embodiments, the platelet concentrate is obtained from one or more single donor platelet units. In some embodiments, the donor platelet units are with or without matching ABO blood type. In some embodiments, the units are volume reduced, washed (e.g., with 0.9% saline), suspended and transferred into a blood product bag. As used herein, the term “concentrate” or “concentrating” refers to the separation of platelets from the bulk of the plasma, whole blood, or other fluid from which it is present. For example, centrifugation, spectrometry, filtration, decanting, gravity settling, or other methods of concentrating platelets from platelet-containing fluids can be used. In some embodiments, when concentrating platelets, an anticoagulant (particularly for centrifugation or gravity settling) can be used along with the source of platelets to prevent clotting during the separation of platelets from other components of the blood, plasma, or other fluid.

In some embodiments, the process further comprises (2) quick freezing the bag containing the concentrated platelet. In some embodiments, the freezing cycle is performed at −40° C. and below. In some embodiments, the concentrated platelet can be stored frozen at −80° C. for up to one year and serves as the starting material for manufacture of the platelet lysate product.

In some embodiments, the process further comprises (3) thawing the frozen, concentrated platelet. In some embodiments, the thawing step is done in a refrigerator at 1-6° C. for 12-15 hours. In some embodiments, thawing process can be carried out using a specialized thawing bath or by thawing frozen pre-lysate in a refrigerator with the pre-lysate units submersed in container of water pre-chilled to 1-6° C.

In some embodiments, the process further comprises (4) centrifuging the thawed, pre-lysate concentrated, and separating the precipitate and debris from supernatant. In some embodiments, a speed of about 4500-5500 RPM (e.g., 4,950 RPM) at a temperature of 1-6° C. is used.

In some embodiments, the process further comprises (5) filtering the supernatant and adjusting the concentration. In some embodiments, the concentration is adjusted to about 5-10×10¹¹ platelet equivalents per 3 liter batch, such as 8-9×10¹¹ platelet equivalents per 3 liter batch. In some embodiments, 0.9% saline is added to the filtered solution to achieve the desired concentration.

In some embodiments, the process further comprises (6) filtering the supernatant again to produce a filtered lysate. In some embodiments, a 0.45μ filter is used.

In some embodiments, the process further comprises (7) freezing the filtered lysate and thawing again. In some embodiments, the filtered lysate is frozen at −30° C. In some embodiments, the frozen, filtered lysate is thawed again. In some embodiments, the filtered lysate is filtered at least once again through a 0.45μ filter. In some embodiments, the filtered lysate is filtered at least twice again through a 0.45μ filter and a 0.2μ filter.

The above-mentioned process can be modified to add, reduce, or change a certain step, as long as a platelet lysate is produced.

The compositions of present disclosure can be used for many purposes, and be formulated according to the intended use.

In some embodiments, a formulation of the present disclosure contains controlled amount of platelet lysates and plasma. In some embodiments, the concentration of platelet lysates in the formulation can be in the range of about 1% to about 10%, such as about 1%, about 2%, about 3%, about 4%, about 5%, about 6%, about 7%, about 8%, about 9%, about 10%. The concentration of plasma in the formulation is from about 0.01% to 1%, such as about 0.01%, about 0.02%, about 0.03%, about 0.04%, about 0.05%, about 0.06%, about 0.07%, about 0.08%, about 0.09%, about 0.1%, about 0.2%, about 0.3%, about 0.4%, about 0.5%, about 0.6%, about 0.7%, about 0.8%, about 0.9%, or about 1%.

In some embodiments, the platelet lysate compositions of the present invention can be formulated to be used directly or depleted of water by process such as freeze drying to provide dehydrated products that may be added to cell cultures or pharmaceutically acceptable excipients and used in therapeutic applications.

In some embodiments, the platelet lysate compositions can be formulated as growth media.

In some embodiments, the growth media of the present disclosure can contain other components including, but are not limited to, amino acids (either essential and/or non-essential amino acids), antibiotics, vitamins, energy resource (e.g., a carbohydrate compound, such as glucose, fatty acids), inorganic compounds (e.g. trace elements, inorganic salts), buffering compounds, nucleosides and bases.

In some embodiments, the growth media can be cell culture media, such as a composition of nutrients used for growing cells over a prolonged period of time.

In some embodiments, the growth media can be those used during expansion phase of the overall cell production process. The expansion phase is the first period of the overall cultivation/production process which is predominantly characterized by high cell growth and less polypeptide production. The expansion phase serves the purpose of expanding the cells, which means generating an adequate number of cells which are in the exponential growth phase to inoculate a production bioreactor.

In some embodiments, the growth media can be cell production media, which are used during production phase of the overall production process. The production phase is a second phase of the overall cultivation/production process which serves the purpose of producing high amounts of product. During the production phase the cells is maintained in viable and productive mode as long as possible.

In some embodiments, the growth media can be used during any stage of a cell culture process.

The growth media can be used in various cell culture processes, such as adherent cell culture, monolayer cell culture, suspension cell culture, discontinuous or continuous cell culture processes (e.g., fed-batch and repeated batch). In some embodiments, the cell culture is used to produce a desired product, such as polypeptides. In some embodiments, the polypeptides can be used to produce pharmaceutical products.

In some embodiments, the polypeptides are antibodies. The antibodies can be monoclonal antibodies (including full length monoclonal antibodies), polyclonal antibodies, multispecific antibodies (e.g., bispecific antibodies), nanobodies, modified antibodies, subunits of antibodies, antibody derivatives, artificial antibodies, combinations of antibodies with proteins and antibody fragments sufficiently long to display the desired biological activity.

In some embodiments, the polypeptides are transmembrane proteins, receptors, hormones, growth factors, proteases, clotting and anti-clotting proteins, inhibitor proteins, interleukins, transport factors, fusion proteins and the like. The cell culture medium can also be used for the production of viruses.

The growth media can be used in cell culture prepared for gene therapy (e.g., ex vivo gene therapy), for stem cell therapy, or for immunotherapy.

The growth media can also be used to prepare medicaments comprising biologically active cells. In some embodiments, such medicaments are suitable for administration by inhalation, by injection, or by implantation.

The growth media of the present invention can be used for the cell culture or tissue culture of any mammal. In some embodiments, a method of culturing cells or tissue can comprise a media comprising the compositions of the present invention with a cell or tissue culture.

In some embodiments, the cells to be cultured include, but are not limited to exocrine secretory epithelial cells, hormone secreting cells, keratinizing epithelial cells, wet stratified barrier epithelial cells, sensory transducer cells, autonomic neuron cells, sense organ and peripheral neuron supporting cells, central nervous system neurons and glial cells, lens cells, metabolism and storage cells, barrier function cells, extracellular matrix cells, contractile cells, blood and immune system cells, germ cells, nurse cells, skin cells, and interstitial cells.

Exocrine secretory epithelial cells include, but are not limited to, salivary gland mucous cell (polysaccharide-rich secretion), salivary gland number 1 (glycoprotein enzyme-rich secretion), von Ebner's gland cell in tongue (washes taste buds), mammary gland cell (milk secretion), lacrimal gland cell (tear secretion), ceruminous gland cell in ear (earwax secretion), eccrine sweat gland dark cell (glycoprotein secretion), eccrine sweat gland clear cell (small molecule secretion), apocrine sweat gland cell (odoriferous secretion, sex-hormone sensitive), gland of Moll cell in eyelid (specialized sweat gland), sebaceous gland cell (lipid-rich sebum secretion), Bowman's gland cell in nose (washes olfactory epithelium), Brunner's gland cell in duodenum (enzymes and alkaline mucus), seminal vesicle cell (secretes seminal fluid components, including fructose for swimming sperm), prostate gland cell (secretes seminal fluid components), bulbourethral gland cell (mucus secretion), Bartholin's gland cell (vaginal lubricant secretion), gland of Littre cell (mucus secretion), uterus endometrium cell (carbohydrate secretion), isolated goblet cell of respiratory and digestive tracts (mucus secretion), stomach lining mucous cell (mucus secretion), gastric gland zymogenic cell (pepsinogen secretion), gastric gland oxyntic cell (hydrochloric acid secretion), pancreatic acinar cell (bicarbonate and digestive enzyme secretion, paneth cell of small intestine (lysozyme secretion), type II pneumocyte of lung (surfactant secretion), and clara cell of lung.

Hormone secreting cells include, but are not limited to, anterior pituitary cells, intermediate pituitary cell, magnocellular neurosecretory cells, gut and respiratory tract cells, thyroid gland cells, Parathyroid gland cells, adrenal gland cells, leydig cell of testes, theca interna cell of ovarian follicle, corpus luteum cell of ruptured ovarian follicle, juxtaglomerular cell, macula dense cell of kidney, peripolar cell of kidney, and mesangial cell of kidney.

Keratinizing epithelial cells include, but are not limited to, epidermal keratinocyte (differentiating epidermal cell), epidermal basal cell (stem cell), keratinocyte of fingernails and toenails, nail bed basal cell (stem cell), medullary hair shaft cell, cortical hair shaft cell, cuticular hair shaft cell, cuticular hair root sheath cell, hair root sheath cell of Huxley's layer, hair root sheath cell of Henle's layer, external hair root sheath cell, and hair matrix cell (stem cell).

Wet stratified barrier epithelial cells include, but are not limited to, surface epithelial cell of stratified squamous epithelium of cornea, tongue, oral cavity, esophagus, anal canal, distal urethra and vagina; basal cell (stem cell) of epithelia of cornea, tongue, oral cavity, esophagus, anal canal, distal urethra and vagina; and urinary epithelium cell (lining urinary bladder and urinary ducts).

Sensory transducer cells include, but are not limited to, auditory inner hair cell of organ of Corti, auditory outer hair cell of organ of Corti, basal cell of olfactory epithelium (stem cell for olfactory neurons), cold-sensitive primary sensory neurons, heat-sensitive primary sensory neurons, merkel cell of epidermis (touch sensor), olfactory receptor neuron, pain-sensitive primary sensory neurons (various types), photoreceptor cells of retina in eye, proprioceptive primary sensory neurons (various types), touch-sensitive primary sensory neurons (various types), type I carotid body cell (blood pH sensor), type II carotid body cell (blood pH sensor), type I hair cell of vestibular system of ear (acceleration and gravity), type II hair cell of vestibular system of ear (acceleration and gravity), and type I taste bud cell.

Autonomic neuron cells include, but are not limited to, cholinergic neural cell (various types), adrenergic neural cell (various types), and peptidergic neural cell (various types).

Sense organ and peripheral neuron supporting cells include, but are not limited to, Inner pillar cell of organ of Corti, outer pillar cell of organ of Corti, inner phalangeal cell of organ of Corti, outer phalangeal cell of organ of Corti, border cell of organ of Corti, Hensen cell of organ of Corti, vestibular apparatus supporting cell, taste bud supporting cell, olfactory epithelium supporting cell, Schwann cell, satellite glial cell (encapsulating peripheral nerve cell bodies), and enteric glial cell.

Central nervous system neurons and glial cells include, but are not limited to, astrocyte (various types), neuron cells (large variety of types, still poorly classified), oligodendrocyte, and spindle neuron.

Lens cells include, but are not limited to, anterior lens epithelial cell, and crystallin-containing lens fiber cell.

Metabolism and storage cells include, but are not limited to, hepatocyte (liver cell), adipocytes (e.g., white fat cell and brown fat cell), and liver lipocyte.

Barrier function cells include, but are not limited to, kidney parietal cell, kidney glomerulus podocyte, kidney proximal tubule brush border cell, loop of Henle thin segment cell, kidney distal tubule cell, kidney collecting duct cell, type I pneumocyte (lining air space of lung cell), pancreatic duct cell (centroacinar cell), nonstriated duct cell (of sweat gland, salivary gland, mammary gland, etc.), principal cell, intercalated cell, duct cell (of seminal vesicle, prostate gland, etc.), intestinal brush border cell (with microvilli), exocrine gland striated duct cell, gall bladder epithelial cell, ductulus efferens nonciliated cell, epididymal principal cell, and epididymal basal cell.

Extracellular matrix cells include, but are not limited to, ameloblast epithelial cell (tooth enamel secretion), planum semilunatum epithelial cell of vestibular system of ear (proteoglycan secretion), organ of Corti interdental epithelial cell (secreting tectorial membrane covering hair cells), loose connective tissue fibroblasts, corneal fibroblasts (corneal keratocytes), tendon fibroblasts, bone marrow reticular tissue fibroblasts, other nonepithelial fibroblasts, pericyte, nucleus pulposus cell of intervertebral disc, cementoblast/cementocyte (tooth root bonelike ewan cell secretion), odontoblast/odontocyte (tooth dentin secretion), hyaline cartilage chondrocyte, fibrocartilage chondrocyte, elastic cartilage chondrocyte, osteoblast/osteocyte, osteoprogenitor cell (stem cell of osteoblasts), hyalocyte of vitreous body of eye, stellate cell of perilymphatic space of ear, hepatic stellate cell (Ito cell), and pancreatic stelle cell.

Contractile cells include, but are not limited to, skeletal muscle cell (e.g., red skeletal muscle cell (slow), white skeletal muscle cell (fast), intermediate skeletal muscle cell, nuclear bag cell of muscle spindle, and nuclear chain cell of muscle spindle), satellite cell (stem cell), heart muscle cells, ordinary heart muscle cell, nodal heart muscle cell, purkinje fiber cell, smooth muscle cell (various types), myoepithelial cell of iris, and myoepithelial cell of exocrine glands.

Blood and immune system cells include, but are not limited to, erythrocyte (red blood cell), megakaryocyte (platelet precursor), monocyte (white blood cell), connective tissue macrophage (various types), epidermal Langerhans cell, osteoclast (in bone), dendritic cell (in lymphoid tissues), microglial cell (in central nervous system), neutrophil granulocyte, eosinophil granulocyte, basophil granulocyte, hybridoma cell, mast cell, helper T cell, suppressor T cell, cytotoxic T cell, natural Killer T cell, B cell, natural killer cell, reticulocyte, blood stem cells and committed progenitors for the blood and immune system (various types)

Germ cells include, but are not limited to oogonium/oocyte, spermatid, spermatocyte, spermatogonium cell (stem cell for spermatocyte), and spermatozoon.

Nurse cells include, but are not limited to, Ovarian follicle cell, sertoli cell (in testis), and thymus epithelial cell.

Interstitial cells include, but are not limited to interstitial kidney cells.

In some embodiments, the cells are stem cells. In some embodiments, the stem cells are pluripotent hematopoietic stem cells, pluripotent mesenchymal stem cells, committed progenitor hematopoietic stem cells, including myeloid, lymphoid and erythroid lineages, committed progenitor mesenchymal stem cells, including cardio, neuro, hepatic, pancreatic, pulmonary and musculo-skeletal cells, connective tissue cells, cord blood and cord tissue-derived stem cells, embryonic stem cells, committed progenitors derived from embryonic stem cells, and induced pluripotent stem cells

In some embodiments, the cells are tumor cells. In some embodiments, the tumor cells are malignant solid tumor cells, solid tumor stem cells, malignant hematological cells and malignant hematological stem cells. In some embodiments, the tumor cells are associated with AIDS-related cancers, adrenocortical cancer, anal cancer, bladder cancer, bowel cancer, brain and central nervous system cancers, breast cancer, carcinoid cancers, cervical cancer, chondrosarcoma, choriocarcinoma, colorectal cancer, endocrine cancers, endometrial cancer, Ewing's sarcoma, eye cancer, gastric cancer, gastrointestinal cancer, genitourinary cancers, glioma, gynecological cancer, head and neck cancer, hepatocellular cancer, Hodgkin's disease, hypopharyngeal cancer, islet cell cancer, Kaposi's sarcoma, kidney cancer, laryngeal cancer, leukemia, liver cancer, lung cancer (e.g., Non-Small Cell Lung Cancer), lymphoma, melanoma, basal cell carcinoma, mesothelioma, myeloma, nasopharyngeal cancer, neuroblastoma, non-Hodgkin's lymphoma, esophagael cancer, osteosarcoma, ovarian cancer, pancreatic cancer, pituitary cancer, renal cell carcinoma, prostate cancer, retinoblastoma, rhabdomyosarcoma, sarcoma, skin cancer, squamous cell carcinoma, stomach cancer, testicular cancer, thymus cancer, thyroid cancer, transitional cell cancer, trophoblastic cancer, uterine cancer, vaginal cancer, Waldenstrom's macroglobulinemia, Wilm's cancer, and leukemia.

In some embodiments, the cells are skin cells. In some embodiments, the skin cells are epidermal cells, including but not limited to keratinocytes, Merkel cells, melanocytes, and Langerhands cells. In some embodiments, the skin cells are dermal cells, including but not limited to dermal fibroblasts, macrophages, and adipocytes.

In some embodiments, the platelet lysate compositions of the present invention are formulated for topical application. The formulation can be topically applied to a subject in need. The subject can be a mammalian subject, such as a human.

In some embodiments, the formulation for topical application further comprises at least one antioxidant. In some embodiments, the antioxidant is selected from the group consisting of vitamin E, idebenone, lycopene, vitamin C, silymarin, resveratrol, pomegranate, genistein, pycnogenol, niacinamide, tocotrienols, coenzyme Q 10, alpha lipoic acid, carnosine, glutathione, Superoxide Dismutase, catalase, plant extracts (e.g., pine bark, grape seed extract, onion extract, blueberry extract, apple extract, green tea extract, and gorgonian extract).

In some embodiments, the antioxidant is Superoxide Dismutase, such as SOD1 (Superoxide Dismutase (Cu—Zn)), SOD2 (MnSOD), or SOD3 (extracellular superoxide dismutase). In some embodiments, the concentration of Superoxide Dismutase in the formulation is about 0.01% to about 10%. In some embodiments, the concentration of SOD1 in the formulation is about 0.05% to about 8%. In some embodiments, the concentration of SOD1 in the formulation is about 0.1% to about 5%.

In some embodiments, the formulation for topical application further comprises at least one additional active skin care agent. The additional active skin care agents include, but are not limited to essential oils, antioxidants, free-radical scavengers, reducing agents, collagen stimulating agents, collagen promoters, soluble collagens, self tanners, anti-acne agents, anti-microbial agents, vitamins, skin protecting agents, skin bleaching agents, skin soothing agents, skin conditioners, skin healing agents, anti-redness agents, anti-swelling agents, de-puffing agents, and substances that can plump the skin and substances that can firm or tone the skin.

The additional active skin care agent can comprise at least one vitamin moiety. In some embodiments, the vitamin moiety is a vitamin C moiety. In some embodiments, the vitamin C moiety is ascorbate or derivatives thereof. In some embodiments, the vitamin C moiety is a water soluble ester of ascorbic acid, including but not limited to sodium ascorbyl phosphate, potassium ascorbyl phosphate, magnesium ascorbyl phosphate and calcium ascorbyl phosphate. In some embodiments, the vitamin C moiety is an anhydrous formulation of ascorbic acid, including but not limited to tetrahexyldecyl ascorbate and ascorbyl tetraisopalmitate.

The additional active skin care agent can comprise a substance that can increase collagen levels, including but not limited to dimethyl sulfone, silymarin and grape (Vitis vinifera) seed extract.

The additional active skin care agent can a substance that can reduce skin inflammation, including but not limited to boswelic acid, dipotassium glycyerhizinate, fucoidan, pomegranate extracts, soy isoflavones, gynostemma, vitamins, provitamin B5, niacinamine, inhiphase (Pueraria lobata root extract), quercetin caprylate, bisabolol and silymarin (Silybum marianum fruit extract).

The additional active skin care agent can a substance that can soothe the skin or reduce skin redness, including but not limited to licorice (Glycyrrhiza glabra) root extract, bisabolol, quercetin caprylate, dipotassium glycyrrhizinate and gatuline (Ranunculus ficaria extract).

The additional active skin care agent can a plant-derived skin care agent, such as those described in U.S. Pat. No. 8,980,344, which is hereby incorporated by reference in its entirety.

Other additional active skin care agents that can be used include, but are not limited to, pearl powder, hexapeptide-10, hydrogenated castor oil, beeswax, allantoin, zinc oxide, isononyl isononanoate, isohexadecane, benzoic acid, bentonite, 1-methylhydantoin-2-imide, acrylates/carbamate copolymer, arbutin, lactic acid, mannitol, octoxynol-9, zinc salt of 1-pyrrolidone carboxylic acid, copper salt of 1-pyrrolidone carboxylic acid, sea salt, cocamidopropyl betaine, citric acid, malic acid, jojoba esters, xanthan gum, caffeine, ectoin and ethylene brassylate.

The formulation for topical application is in a topical cream, a topical serum, or a spray mist.

The formulation for topical application can further comprise at least one emollient and/or thickening agent. The emollient and/or thickening agent can be natural, synthetic, or semi-synthetic. The emollient and/or thickening agent can be selected from polysaccharides, proteins, alcohols, silicones, fatty acids (e.g., saturated or unsaturated fatty acids, and triglyerides thereof) and waxes. As used herein, an emollient agent is a waxlike, lubricating agent that prevents water loss and has a softening and soothing effect on skin. None-limiting examples of emollient agents include plant oils, mineral oil, shea butter, cocoa butter, petrolatum, fatty acids, triglycerides, benzoates, myristates, palmitates, and stearates. As used herein, a thickener enhances the consistency, volume and viscosity of cosmetic products, thereby providing more stability and better performance. While some thickeners have also emulsifying or gelling properties, the majority of thickeners have the ability to retain water on the skin and act therefore as moisturizers. In some embodiments, the emollient and/or thickening agent comprises linoleic acid or derivatives thereof. In some embodiments, the concentration of linoleic acid or derivatives thereof in the formulation is about 0.1% to about 50%. In some embodiments, the concentration of linoleic acid or derivatives thereof in the formulation is about 1%, about 2%, about 3%, about 4%, about 5%, about 5%, about 6%, about 7%, about 8%, about 9%, about 10%, about 15%, about 20%, about 25%, about 30%, about 35%, about 40%, about 45%, or about 50%. In some embodiments, the concentration of linoleic acid or derivatives thereof in the formulation is about 1% to about 40%. In some embodiments, the concentration of linoleic acid or derivatives thereof in the formulation is about 1% to about 2% to about 30%.

The present invention also provides skin care products comprising the formulations for topical application described herein.

The present invention also provides a process of preparing the formulations described herein, wherein the process comprises mixing a platelet lysate composition of the present application with other ingredients.

The present invention also provides methods of promoting cell growth. In some embodiments, the methods promote growth of skin cells. In some embodiments, the skin cells are dermal fibroblasts. Dermal fibroblasts are cells within the dermis layer of skin which are responsible for generating connective tissue. In some embodiments, the methods comprise topical application of a formulation of the present invention to the skin of a subject in need.

In some embodiments, the formulations of the present invention can be applied in a suitable frequency and duration. In some embodiments, the formulations can be applied once, twice, three times, four times, five times, six times, or more daily. In some embodiments, the formulations can be applied daily, every two days, every three days, every week, or a longer period of time. In some embodiments, the formulation is applied for a period of about 1-10 days, about 1-20 days, about 1-30 days, about 1-40 days, about 1-50 days, about 1-60 days, about 1-70 days, about 1-80 days, about 1-90 days, about 1-100 days, or more. In some embodiments, the application is followed by about 1-5 days, about 5-10 days, about 10-20 days, about 20-30 days of non-application.

In some embodiments, the formulations are applied to subjects with wound, scars and/or wrinkles. In some embodiments, the types of scar can be hypertrophic, keloid, atrophic, or sunken scarring.

In some embodiments, the formulations are applied to subjects at risk of developing wound, scars and/or wrinkles. In some embodiments, the formulations can be applied to the skin of a subject having, had, or at risk of having trauma to the skin, such as physical wounding, burning, freezing, radiation, chemical erosion, surgery, insects biting, pathogen infection (e.g., shingles, folliculitis, keratosis pilaris, impetigo, cold sores, monkeypox, smallpox, leishmaniasis, AIDS), inflammation, autoimmune (e.g., lupus, scleroderma), pruritus, skin cancer (e.g., melanoma), Dupuytren's contracture, acne, sunburn, moles, lichen sclerosus, molluscum contagiosum, psoriasis, etc.

In some embodiments, the methods comprise applying a formulation of the present invention in combination with one or more other agent that promotes skin cell growth.

The present invention also provides methods of treating wounds, scars and/or wrinkles. In some embodiments, the methods improve wound healing and/or reducing scar or wrinkle formation. In some embodiments, the methods comprise topical application of a formulation for topical application to skin of a subject. In some embodiments, the formulation is applied to facial, neck, hands, feet skin of the subject or other parts of the body. In some embodiments, the subject is a human. In some embodiments, the methods comprise rubbing or applying the formulation of the invention to the skin of a subject, with optional massaging of the formulation on and/or into the skin. As used herein, the term “treating” or “treatment” as used herein refer to an approach for obtaining beneficial or desired results including clinical results, and may include even minimal changes or improvements in one or more measurable markers of the disease or condition being treated. A treatment is usually effective to reduce at least one symptom of a condition, disease, disorder, injury or damage. Exemplary markers of clinical improvement will be apparent to persons skilled in the art. Examples include, but are not limited to, one or more of the following: decreasing the severity and/or frequency one or more symptoms resulting from the disease or condition, diminishing the extent of the disease or condition, stabilizing the disease or condition (e.g., preventing or delaying the worsening of the disease), delay or slowing the progression of the disease or condition, ameliorating the disease state, decreasing the dose of one or more other medications required to treat the disease or condition, and/or increasing the quality of life, accelerating the progress of healing, etc. The term “treating” or “treatment” also refers to an approach that prevents the happening of one or more symptoms, or reduces the frequency of its happening.

In some embodiments, the methods improve a parameter of wound healing or scar treatment in a subject. The parameters will be known in the field of wound/scar care. In some embodiments, the parameter is selected from the group consisting of speedier healing, increased scar tissue organization, less visible scar tissue thickness, lower epidermal thickness, lower dermal thickness, softened scar contours, decreased scar depth, decreased scar area, etc. and combinations thereof.

In some embodiments, the methods improve a parameter of wrinkle treatment in a subject. The parameters will be known in the field of skin care. In some embodiments, the parameter is selected from the group consisting of transepidermal water loss (TEWL), elasticity, pliability, wrinkle severity grades, self-assessment of satisfaction, etc. and combinations thereof. Additional parameters can be found in Hwang et al. (Ann. Dermatol. 25(4):445-453, 2013), Fu et al. (British Journal of Dermatology, 162:647-654, 2010), and Jaffary et al. (J. Res. Med. Sci. 18(11):970-975, 2013), each of which is hereby incorporated by reference in its entirety.

The methods of the present invention provide a clinically relevant, statistically significant result. In some embodiments, the statistically significant treatment effect is determined based on one or more standards or criteria provided by one or more regulatory agencies in the United States, e.g., FDA or other countries. In some embodiments, the statistically significant treatment effect is determined based on results obtained from regulatory agency approved clinical trial set up and/or procedure, or parameters as described herein.

The statistically significant treatment effect is determined based on a subject population of at least 10, 20, 30, 40, 50, 100, 200, 300, 400, 500, 600, 700, 800, 900, 1000, or more. In some embodiments, the statistically significant treatment effect is determined based on data obtained from randomized and double blinded clinical trial set up. In some embodiments, the statistically significant treatment effect is determined based on data with a p value of less than or equal to about 0.05, 0.04, 0.03, 0.02 or 0.01. In some embodiments, the statistically significant treatment effect is determined based on data with a confidence interval greater than or equal to 95%, 96%, 97%, 98% or 99%.

The formulations of the present invention can be combined with other wound, scar or wrinkle treatments, including but not limited to chemical peels, filler injections, laser treatment, radiotherapy, dressing, steroids, surgery, dermabrasion, massage, silicone gel sheet treatment, laser, or cosmetic/medical makeup.

Compositions of the present invention can be used in therapeutic or cosmetic treatments.

In some embodiments, compositions of the present invention are used to induce differentiation of stem cells. In some embodiments, the compositions are used to induce endothelial differentiation of bone marrow mesenchymal stem cells.

In some embodiments, compositions of the present invention are used in wound healing and/or reducing inflammation, such as those described in Knighton et al. (Stimulation of repair in chronic, nonhealing, cutaneous ulcers using platelet-derived wound healing formula. Surg Gynecol Obstet 1990; 170:56-60), Steed et al. (Randomized prospective double-blind trial in healing chronic diabetic foot ulcers. CT-102 activated platelet supernatant, topical versus placebo. Diabetes Care 1992; 15:1598-604.), Knighton et al. (Classification and treatment of chronic nonhealing wounds. Successful treatment with autologous platelet-derived wound healing factors (PDWHF). Ann Surg 1986; 204:322-30.), Atri et al. (Use of homologous platelet factors in achieving total healing of recalcitrant skin ulcers. Surgery 1990; 108:508-12.), Glover et al. (A 4-year outcome-based retrospective study of wound healing and limb salvage in patients with chronic wounds. Adv Wound Care 1997; 10:33-8.), Margolis et al. (Effectiveness of platelet releasate for the treatment of diabetic neuropathic foot ulcers. Diabetes Care 2001; 24:483-8.), Keyser (Diabetic wound healing and limb salvage in an outpatient wound care program. South Med J 1993; 86:311-7.), each of which is herein incorporated by reference in its entirety.

In some embodiments, the compositions are used to treat injured nerves. In some embodiments, the compositions are used to treat back pain, such as spinal disc pain.

In some embodiments, compositions of the present invention are used in cell therapy. The term “cell therapy” refers to the therapy in which cellular material is injected into a patient. The compositions can be used to culture cells to be injected.

In some embodiments, compositions of the present invention are used as a source for anti-inflammatory factors, or growth factors such as platelet-derived growth factor (PDGF), platelet factor 4, transforming growth factor-β, platelet-derived angiogenesis factor, and platelet-derived epidermal growth factor. The combination of PDGF, platelet-derived angiogenesis factor, transforming growth factor-β, and possibly platelet-derived epidermal growth factor enhances granulation tissue growth while stimulating angiogenesis, fibroplasia, and extracellular matrix synthesis. Platelet factor 4 and PDGF attract neutrophils for host defense, and the combination of PDGF and platelet-derived epidermal growth factor stimulates epithelialization.

In some embodiments, compositions of the present invention are used in topical and injectable applications. In some embodiments, the compositions are used to produce ocular drugs to treat dry-eyes.

In some embodiments, the compositions of the present invention are used in the treatment of conditions related to chronic ulcers, bone, tendon, never regeneration, tissue engineering, and cell therapy.

In some embodiments, the compositions of the present invention are used in skincare products. In some embodiments, the skincare products comprising the compositions of the present invention provide for anti-wrinkling effect, removing pigmentation, reducing skin lines, increasing skin elasticity/tightening skin, stimulating the renewal of dermal cells, rebuilding skin tissues, ameliorating amyloidosis, ameliorating keratosis pilaris and/or prevention/reduction of scar tissue. In some embodiments, the compositions of the present invention are used to promote the formation of new fibroblasts, which result in healthier, restored skin.

The following examples illustrate various aspects of the invention. The examples should, of course, be understood to be merely illustrative of only certain embodiments of the invention and not to constitute limitations upon the scope of the invention.

### Examples

### Example 1. Manufacturing Process of Human Platelet Lysate (hPL) Products

An exemplary manufacturing process for hPL product of the present disclosure is described in the following steps as follows:

A. Preparation of plasma reduced, washed frozen platelet concentrate (pre-lysate).
1. The first step is obtaining two or more single donor platelet units with matching ABO blood type. These units are volume reduced and washed with 0.9% saline. The washing is performed in a Cobe 2991 cell washer using one volume reduction and 1 washing cycle.
2. After processing on the cell washer, the platelets are mixed on an orbital mixer for 30 minutes at a speed of 180 rotations per minute (RPM). If full resuspension of the platelets is not achieved, the platelet mix is massaged by hand on the bench top until it is fully suspended.
3. The platelet bags are then drained by gravity, volume reduced, and the washed platelet concentrate is transferred into a 300 mL blood product transfer bag.
4. After transfer, any residual air in the blood product bag containing the washed platelet concentrate is expressed, tubing sealed, and the unit is placed into a sealable plastic bag and then into a blast freezer for a quick freezing cycle. For the best results the freezing cycle should be performed at −40° C. and below.
5. After initial freezing, the washed platelet concentrate pre-lysate is stored frozen at −80° C. for up to one year and serves as the starting material for GMP manufacture of the hPL product.

B. First filtration and pooling of the hPL into a standardized batch.
1. The manufacturing process for human platelet lysate consists of collecting twelve frozen pre-lysate units of matching ABO blood type and thawing them in a refrigerator at 1-6° C. for 12-15 hours. For shorter thaw times, the thawing process can be carried out using a specialized thawing bath or by thawing frozen pre-lysate in a refrigerator with the pre-lysate units submersed in container of water pre-chilled to 1-6° C. These methods enable thawing within 3 hours.
2. After thawing the twelve bags of pre-lysate concentrate are centrifuged at a speed of 4,950 RPM at a temperature of 1-6° C. for at least 2 hours.
3. After centrifugation the bags are divided with large sealing clips to separate precipitate and debris from supernatant.
4. The bags are then inverted with the bag ports downward and the precipitate and debris above the sealing clip.
5. Each bag is spiked with one 0.65μ filter and the supernatant is drained by gravity into an attached bag.
6. The twelve filtered units are pooled into one batch by combining in one 3 Liter transfer pack bag.
7. The solution volume for the pooled hPL batch is adjusted by the addition of 0.9% saline to achieve a concentration of 84×10¹¹ platelet equivalents per 3 liter batch. Each hPL batch contains the equivalent of approximately 24 units of single donor platelet concentrate.

C. Secondary filtration of the hPL 3 liter batch intermediate.
1. A second filtration of the in-process hPL 3 liter batch is carried out with a 0.45μ filter at room temperature in an aseptic work space and laminar flow hood. The 0.45μ filtration may be carried out with a Thermo-Nalgene vacuum assisted filtration set with 0.45μ filter or other comparable device.
2. The 3 Liter bag of in-process hPL intermediate is drained by gravity into the filtration set, filling the top chamber. Multiple 1 Liter filtration/bottle sets may be used to complete the filtration of the entire 3 Liter batch.
3. After filling the bottom 1 Liter receiver bottle with filtrate, the bottles are capped and set aside for filling of final product bottles (packaging).

D. Freezing, Thawing and Final Filtration
1. The 0.45μ filtered lysate is frozen at −30° C.
2. At a later time, the batch is thawed at 1-6° C.
3. The thawed lysate is filtered again through a 0.45μ filter
4. The thawed, filtered lysate is then filtered again through a 0.2μ filter

E. Final hPL product packaging
1. The pooled and filtered final product is transferred into final product bottles (Nalgene) for sales and distribution.
2. Clearsate™ brand labels, and product labels, generated with our blood product management computer system are then applied to the product bottles.
3. The 3 Liter batch of filtered hPL final product is transferred into final product bottles.
4. A sample of the each batch of hPL is sterility tested for aerobic and anaerobic bacterial and fungal contamination and held in quarantine until sterility testing is completed.
5. The Certificate of Analysis and Release is signed off after Quality Assurance review is performed.
6. The final product may be stored frozen at ≦−20° C. until use.

### Example 2. Growth-Promoting Activity of Human Platelet Lysate Composition

For the purpose of describing the advantages of the present disclosure, a number of nonlimiting Examples are stated below. In all examples the platelet lysate having plasma in the present disclosure is designated by CS+1W. The FBS used was supplied by the American Tissue Culture Collection (ATCC®).

Growth effect results of media supplemented with 5% human platelet lysate with the controlled addition of human plasma (CS+1W) were compared to 5% FBS. All studies were carried out in DMEM/F12 media (ATCC®), and adherent human umbilical cord mesenchymal stem cells (MSC) were purchased from ATCC® and plated at a final concentration of 5000 cells/well in 24 well tissue culture plates containing 1 mL of test media. Quadruplicate samples were collected by trypsinization at all timepoints (48, 72, 96, 120 and 144 hrs post-plating), and cell counts were determined using a Sysmex cell counter. Cell counts at each timepoint are presented as the mean±SEM.

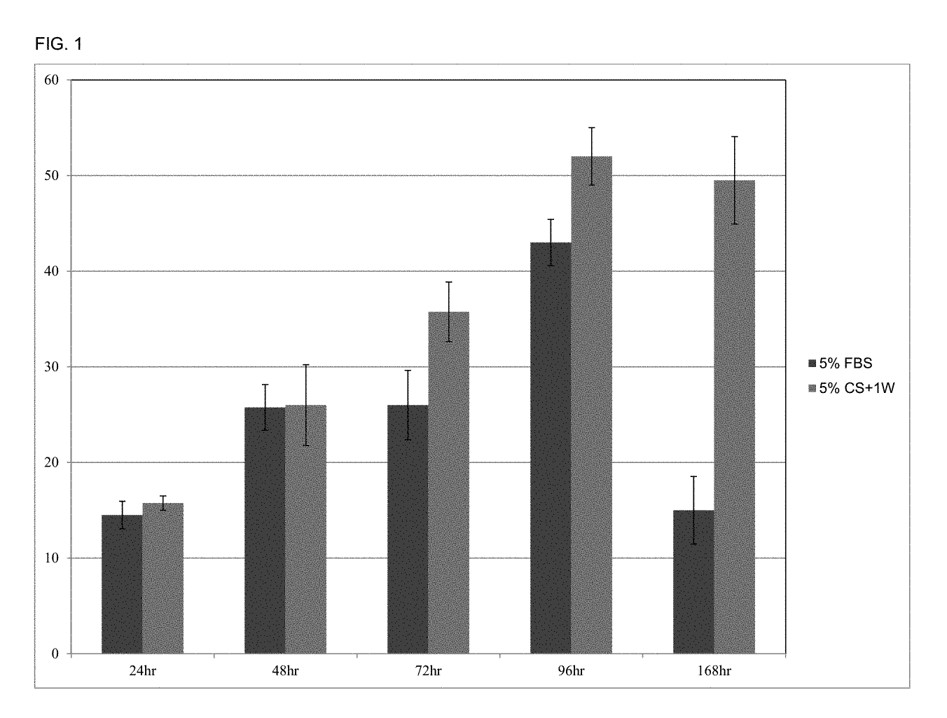

As shown in FIG. 1, media prepared with a final concentration of 5% human platelet lysate containing human plasma typically had greater growth-promoting activity on MSCs at all time points as well as maintaining higher cell densities per well at the 120-144 hr time points when compared to 5% FBS.

### Example 3. Maintaining Higher Cell Densities Activity of Human Platelet Lysate Composition

The growth effect results of media containing 2% human platelet lysate with the controlled addition of human plasma (CS+1W) were compared to 2% human plasma-free platelet lysate (Clearsate™) or 2% FBS. All studies were carried out in DMEM/F12 media (ATCC®), and adherent human umbilical cord mesenchymal stem cells (MSC) were purchased from ATCC® and plated at a final concentration of 5000 cells/well in 24 well tissue culture plates containing 1 mL of test media. Quadruplicate samples were collected by trypsinization at all timepoints (48, 72, 96, 120 and 144 hrs post-plating), and cell counts were determined using a Sysmex cell counter. Cell counts at each timepoint are presented as the mean±SEM.

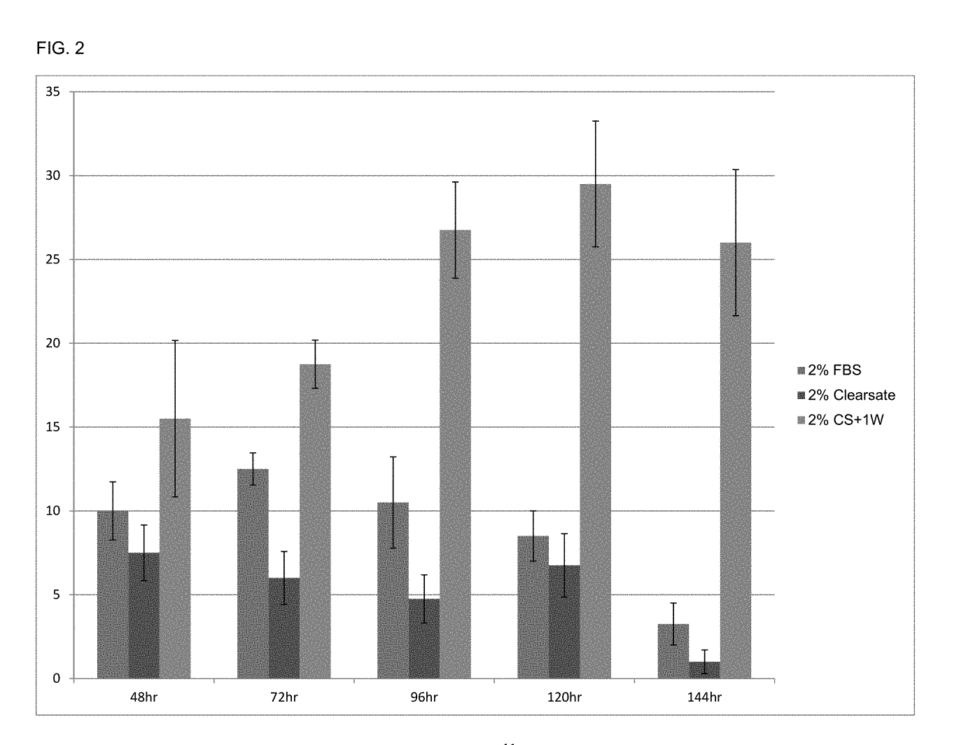

As shown in FIG. 2, media prepared with a final concentration of 2% human platelet lysate containing human plasma typically had greater growth-promoting activity on MSCs at all time points as well as maintaining higher cell densities per well at the 120-144 hr time points when compared to 2% FBS or 2% Clearsate containing no plasma.

### Example 4. Shortening Doubling Time Activity of Human Platelet Lysate Composition

The doubling time results of media supplemented with human platelet lysate with the controlled addition of human plasma (2% CS+1W) were compared to 2% human plasma-free platelet lysate (orig. Clearsate), 2% FBS and two commercial human platelet lysate products (2% Cook and 2% Mill Creek). All studies were carried out in F12K media (ATCC), and A549 adherent human lung adenocarcinoma cells purchased from ATCC and plated at a final concentration of 5000 cells/well in 24 well tissue culture plates containing 1 mL of test media. Quadruplicate samples were collected by trypsinization at all timepoints (48, 72, 96 hrs post-plating), and cell counts were determined using a Sysmex cell counter. Cell doubling times were calculated using a Least Squares Fitting-Exponential model (Roth V. 2006, available from www.doubling-time.com).

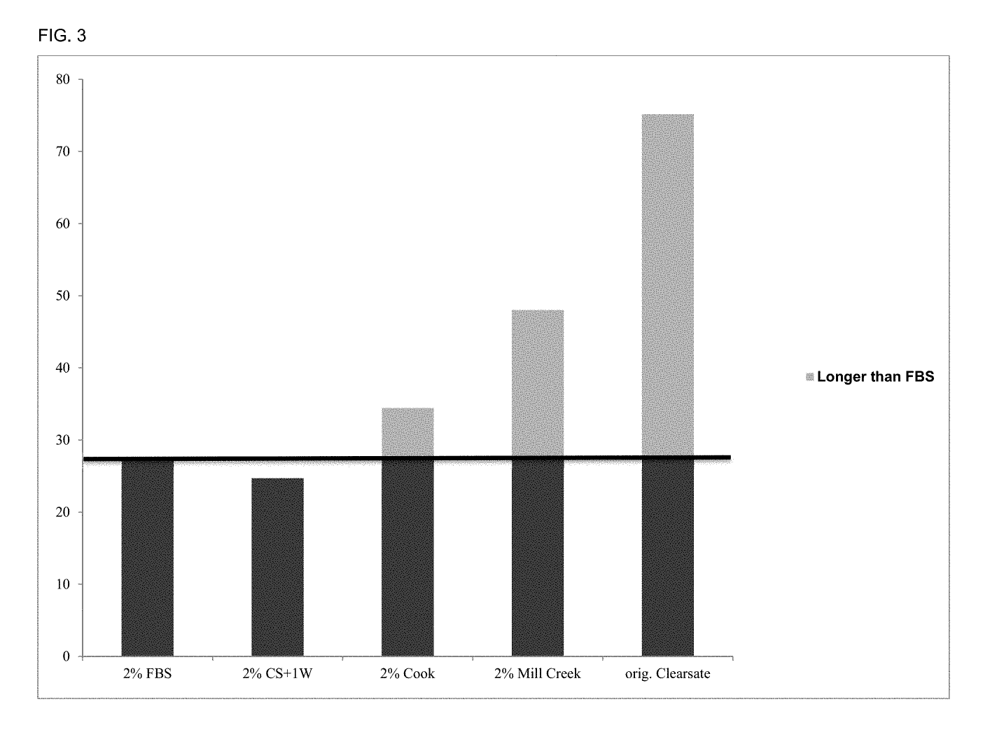

As shown in FIG. 3, media prepared with a final concentration of 2% human platelet lysate containing human plasma (2% CS+1W) typically resulted in shorter doubling times for A549 lung adenocarcinoma cells when compared to original Clearsate, Mill Creek human platelet lysate, Cook Platelet lysate and FBS.

### Example 5. Growth-Promoting Activity on Primary Human Dermal Fibroblasts

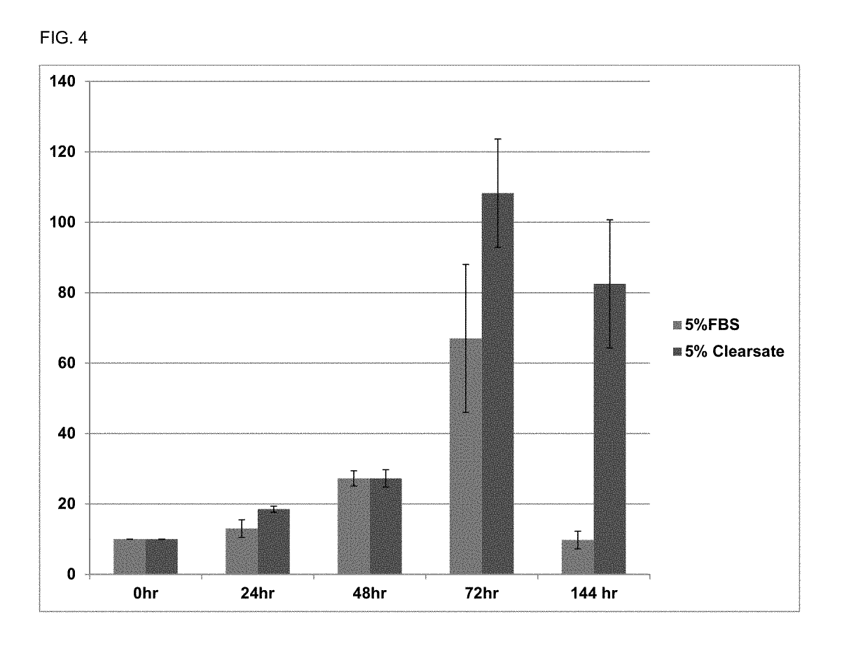

FIG. 4 depicts growth-promoting activity on primary human dermal fibroblasts of media prepared with a final concentration of 2% human platelet lysate containing human plasma, and its ability of maintaining higher cell densities, when compared to 2% FBS or 2% Clearsate containing no plasma.

Frozen primary human dermal fibroblasts were purchased from the American Type Culture Collection (ATCC). The cells were thawed and plated at an initial density of 10,000 cells/well in 24 well tissue culture plates (Corning). Cells were incubated in the presence of 2.5 mL of DMEM/F12 Media (Gibco) supplemented either with 5% (vol/vol) FBS or 5% human platelet lysate for up to 144 hours at 37° C. in a humidified 5% CO2 incubator. Cells were harvested at 24, 48, 72 and 144 hours by trypsinization (Gibco), and cell counts carried out using a Sysmex cell counter. All timepoints were carried out in triplicate and the mean and SEM plotted at each timepoint. At the 72 hour timepoint the human platelet lysate cultured dermal fibroblasts had a 37% greater number of cells than in the FBS cultured cells, and at 144 hours the human platelet lysate cultured dermal fibroblasts had >300% greater number of cells than in the FBS cultured cells. Importantly, there was no obvious cell death in the human platelet lysate cultured cells at 144 hours, while there was a significant decrease in cell number in the FBS cultured cells at 144 hours, indicating superior long term culture capacity on dermal fibroblasts for the human platelet lysate media compared to the FBS media.

Unless defined otherwise, all technical and scientific terms herein have the same meaning as commonly understood by one of ordinary skill in the art to which this invention belongs. Although any methods and materials, similar or equivalent to those described herein, can be used in the practice or testing of the present disclosure, the preferred methods and materials are described herein. All publications, patents, and patent publications cited are incorporated by reference herein in their entirety for all purposes.

The publications discussed herein are provided solely for their disclosure prior to the filing date of the present application. Nothing herein is to be construed as an admission that the present disclosure is not entitled to antedate such publication by virtue of prior invention.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

1. A mammalian platelet lysate composition comprising an amount of lysed mammalian platelets and mammalian plasma, wherein the concentration of mammalian plasma in the composition is from about 1% to about 10%.

2.-3. (canceled)

4. The composition of claim 1, wherein the composition is substantially free of mammalian platelet membranes.

5.-6. (canceled)

7. The composition of claim 1, wherein the composition is substantially free of any heparin.

8. The composition of claim 1, wherein the concentration of platelet lysates in the composition is from about 90% to about 99%.

9.-11. (canceled)

12. The composition of claim 1, wherein the composition has at least 50% of increased growth-promoting activity on cells when used as a supplement in a cell culture media, compared to a plasma-free platelet lysate composition, and/or at least 20% of increased growth promoting activity on cells compared to fetal bovine serum (FBS) at the same concentration.

13. The composition of claim 1, wherein the composition is capable of maintaining at least 80% of increased cell density at the 120-144 hr time points when used in a cell culture media compared to a plasma-free platelet lysate composition, or fetal bovine serum (FBS) at the same concentration.

14. The composition of claim 1, wherein the composition shortens cell growth doubling time when used as a supplement in a cell culture media compared to a cell culture media without the composition.

15.-16. (canceled)

17. A formulation comprising the platelet lysate composition of claim 1.

18. The formulation of claim 17, wherein the concentration of platelet lysates in the formulation is from about 1% to about 10%.

19. A formulation comprising mammalian platelet lysate and mammalian plasma, wherein the platelet lysate concentration in the formulation is from about 1% to about 10%, and the plasma concentration in the medium is from about 0.01% to 1%.

20. (canceled)

21. The formulation of claim 19, wherein the formulation is suitable for being used as a growth medium or for topical application to skin of a subject.

22. The formulation for topical application of claim 21, wherein the formulation further comprises at least one antioxidant.

23. (canceled)

24. The formulation for topical application of claim 21, wherein the formulation further comprises at least one additional active skin care agent.

25.-27. (canceled)

28. The formulation for topical application of claim 21, wherein the formulation is in a topical cream or a topical serum.

29. The formulation for topical application of claim 21, wherein the formulation further comprises at least one emollient and/or thickening agent.

30.-31. (canceled)

32. The formulation for topical application of claim 21, wherein the formulation further comprises Superoxide Dismutase (Cu—Zn) (SOD1), a vitamin C moiety, and linoleic acid.

33. A method of promoting cell growth, comprising contacting cells with the formulation of claim 19.

34. (canceled)

35. The method of claim 33, wherein the cells are stem cells.

36. (canceled)

37. The method of claim 33, wherein the cells are tumor cells.

38.-40. (canceled)

41. The method of claim 33, wherein the cells are skin cells.

42. The method of claim 41, wherein the skin cells are dermal fibroblasts.

43. (canceled)

44. The method of claim 33, wherein the cells are skin cells and the formulation is applied topically.

45. The method of claim 44, wherein the cell growth is associated with wound healing, reduce scar formation, and/or reduce wrinkle formation.

46. The method of claim 44, wherein the topical formulation is applied to the skin of a subject having a trauma to the skin, or a subject at risk of having a trauma to skin.
